(19) United States
(12) Patent Application Publication
(10) Pub. No.: US 2005/0075666 A1
(43) Pub. Date: Apr. 7, 2005

(54) Soft tissue suspension device

(76) Inventors: Corey S. Maas, Sausalito, CA (US); Sheldon S. Kabaker, Oakland, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/678,594

(22) Filed: Oct. 3, 2003

(51) Int. Cl. A61B 17/06

(52) U.S. Cl. 606/224

(57) ABSTRACT

A surgical device for suspending soft tissue in a procedure for elevation of the malar fat pad is constructed from a pair of shafts that are fused to the ends of two sutures, such that both sutures are fused at one end to a common shaft at the proximal end of the shaft, and at their other ends to a second common shaft at its proximal end. The distal ends of both shafts are free for insertion into incisions in the patient, and are blunt-tipped to avoid injuries to nerves or blood vessels. One of the sutures, termed the suspension suture, is threaded through an anchor graft, to engage and elevate the malar fat pad, the graft being preferably ring-shaped to allow ingrowth of soft tissue.

### BACKGROUND OF THE INVENTION

1. Field of the Invention

This invention resides in the field of cosmetic surgery of the face, and in particular, surgery for reversing the effects of aging.

2. Description of the Prior Art

Meloplication of the malar fat pads is a cosmetic surgery technique developed in the late 1990's to reverse midface ptosis, a manifestation of aging in the face. Midface ptosis is caused by a downward migration of the malar fat pads over time due to gravity. A malar fat pad is located in each cheek on the outer side of the nasolabial and melolabial folds, which are the folds extending from the outer edge of the nostrils to the extremities of the lips and downward. The sagging of the malar fat pads produces a deepening of the nasolabial and labiomental folds and a hollowness in the midfacial and infraorbital areas. Reversal of this sagging produces the effect of a more youthful appearance in the midface area and although to a lesser extent, a reduction of the fullness at the jowl. Details of the technique are set forth in two published papers: Sasaki, G. H., et al., “Meloplication of the Malar Fat Pads by Percutaneous Cable-Suture Technique for Midface Rejuvenation: Outcome Study (392 Cases, 6 Years' Experience),” Plastic and Reconstructive Surgery, August 2002, pages 635-654; and Keller, G. S., et al., “Elevation of the Malar Fat Pad With a Percutaneous Technique,” Arch. Facial Plast. Surg., vol. 4, January-March 2002, pages 20-25.

According to the procedure described in these papers, a dot incision is made on the nasolabial fold, and a series of needles and sutures are used to place a permanent suspension suture which is looped through an anchor graft. Both ends of the suture are inserted to place the suture in a U-shaped configuration through the soft tissue, and once in place the suture is guided in a direction perpendicular to the nasolabial line to a position at which the anchor graft engages the soft tissue and malar fat pad. A second series of needles and sutures, identical to the first, is then inserted in the same way into a second dot incision on the nasolabial fold at a site approximately 1 cm from the first dot incision. Both suspension systems, i.e., all four suture ends, are drawn through a linear incision above the hair line for manipulation by the surgeon who then applies tension to both suspension systems at these protruding ends to produce a broad repositioning of the malar fat pad. The suture ends are then fixed to the deep temporal fascia to stabilize the soft-tissue repositioning. The anchor grafts are cup-shaped with holes through which the suspension sutures are threaded, and the graft material is GORE-TEX®, a registered trademark of W.L. Gore & Associates (Flagstaff, Ariz., USA). GORE-TEX is a microporous fluorocarbon polymer.

Elevation of the malar fat pad is typically accompanied by other effects that likewise tend to result from the tension applied by these sutures. These include dimpling at the nasolabial line, flattening of the anterior midcheek, and upward distortion of the upper lip. Removal of these undesired effects is achieved by using a separate suture which is inserted together with, and in approximately the same configuration as, each suspension suture. This second suture, which can be termed a “tissue-release suture” and is not joined to the anchor graft, is manipulated by the surgeon in a back-and-forth cutting action to release the soft tissue from the malar fat pad. This is done until all signs of dimpling, flattening, and distortion due to the suspension system are eliminated. A third part of the procedure in many cases is a guide suture which is looped through the anchor graft although extending in the opposite direction with its ends protruding through the dot incision rather than extending upward toward the temporal area. The protruding ends of the guide suture are manipulated by the surgeon after the anchor graft is inserted, to assure that the anchor graft in the desired orientation cupping the malar fat pad.

The procedure as presently known suffers a number of disadvantages. For example, the insertion of the suspension and tissue-release sutures is achieved by Keith needles, which are straight needles with sharp cutting points, similar to knife blade edges, at one end and an eye at the other end. To assemble the sutures, the surgeon, or an assistant to the surgeon, must attach the ends of both sutures to the Keith needles by threading the sutures through the eyes of the Keith needles such that each Keith needle is secured to both sutures. This is a tedious procedure since it involves threading two sutures through the eye of a single needle, and it must be done separately for each surgery. For one suspension system, therefore, one pair of suture ends must be threaded through each needle eye and kept from pulling back through while the placement is being performed. Two suspension systems require the threading of eight suture ends (in pairs) through the eyes of four needles. This contributes significantly to the time required for the procedure as well as its cost. The handling of all these components raises a risk of contamination and all must therefore be absolutely sterile. Furthermore, threading and securement after threading are difficult, and if the ends of the sutures are not properly secured, they may slip out of the eyes before the procedure is completed. Still further, the cutting points at the leading ends of the Keith needles present a risk of damage to nerves and blood vessels in the soft tissues that the needles must penetrate during insertion of the sutures, as well as in the facial skin itself. A further disadvantage is that Keith needles are generally of limited length (2-4 inches) which makes the procedure difficult on some patients. Also, orientation of the cup-shaped anchor adds to the time required for completion of the procedure and to the risk of an unsuccessful procedure if proper orientation is not achieved. Finally, the knot by which the suspension suture is anchored to the thick connective tissue in the hairline is neither adjustable nor reversible.

These and other shortcomings and disadvantages of the procedure are addressed by the present invention.

### SUMMARY OF THE INVENTION

This invention resides in a surgical device designed for suspending soft tissue to correct midface ptosis in procedures that essentially or approximately follow the surgical steps outlined above, the device being pre-formed so that no assembly is required at the time of the procedure and many of the risks of the prior art methods are either reduced or eliminated. The pre-formed device of this invention includes two rigid, straight shafts that are integrally joined to the ends of two sutures, each shaft being joined to two sutures to allow the double U-shaped configuration described above, with an anchor graft attached to one of the sutures between the ends of that suture. The two sutures are the suspension suture and the tissue-release suture, respectively, and the integral connection between the suture ends and the shafts is achieved by fusing. The terms “fuse,” “fusing,” and “fused” are used herein to mean a permanent joinder of the suture with the shafts to form a unit such that the sutures cannot be disengaged during use. The joinder is achieved by manufacturing procedures and not by threading the end of a suture through an eye and tying the threaded suture into a loop passing through the eye. Preferably, the shafts do not terminate in eyes at all.

The shaft portions of the surgical device replace the Keith needles of the prior art, and preferably differ from Keith needles in various ways. For example, the shafts are preferably tapered and blunt tipped at the ends opposite the ends to which the sutures are fused, to avoid puncture injuries to nerves and blood vessels. The shafts are also preferably of a length that exceeds the typical length of a Keith needle, thereby rendering the device more versatile than those assembled from Keith needles.

In further preferred embodiments of the invention, the cup-shaped anchor graft of the prior art is replaced by a toroidal or ring-shaped member, whose central passage both accommodates the suspension suture and allows the passage of soft tissue regrowth as the tissue that was severed during the procedure grows back during healing. Regrowth through the passage provides particularly strong anchoring of the graft into the soft tissue.

In still further embodiments, a small biocompatible clasping device is included in the system to allow the surgeon to secure the free ends of the suspension sutures in the fascia or connective tissue without tying a knot. The applied tension can then be adjusted at any time without having to remove new suspension sutures and replace them with new ones.

These and other features, embodiments, objects and advantages of the invention will be clearer from the detailed description offered below.

### BRIEF DESCRIPTION OF THE DRAWING

FIG. 1 is a schematic representation of a soft tissue suspension device in accordance with the present invention.

FIG. 2 is a drawing of the suspension suture of the device of FIG. 1 with a clasping device joining the ends of the suture.

### DETAILED DESCRIPTION OF THE INVENTION AND PREFERRED EMBODIMENTS

While the invention is susceptible to a range of constructions and embodiments, the concepts and principles that define the invention are best understood by a detailed examination of a specific example. One such example is shown in the drawings and described below.

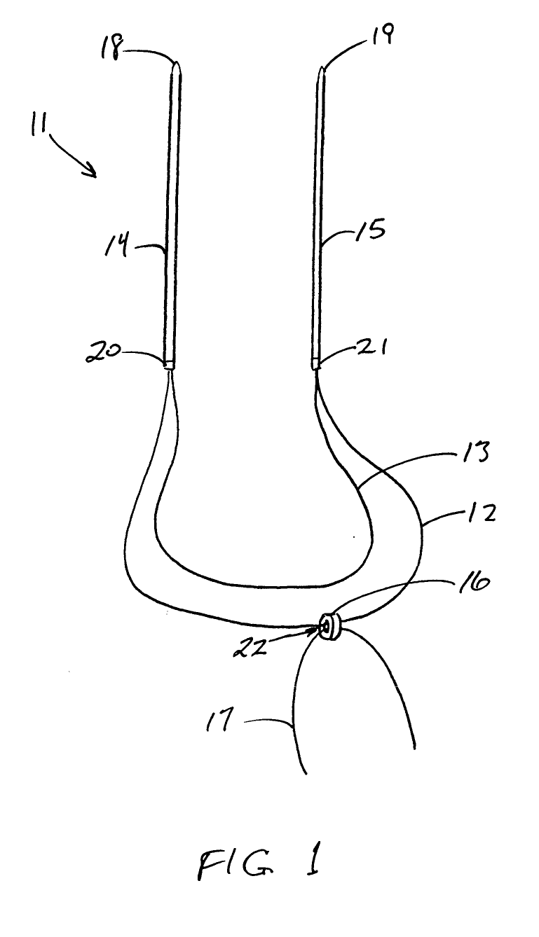

The surgical device 11 shown in FIG. 1 includes a suspension suture 12, a tissue-release suture 13, two shafts 14, 15, a ring-shaped anchor graft 16, and an optional safety suture 17. Any of the sutures of this device can be either monofilament or multifilament sutures, and absorbable or nonabsorbable. Examples of typical suture materials are PROLENE® (polypropylene), a non-absorbable monofilament material and a registered trademark of Ethicon, Inc., a Johnson and Johnson company, VICRYL® (polyglactin), an absorbable braided suture material and a registered trademark of Ethicon, Inc., DEXON®, an absorbable braided suture material and a registered trademark of Ethicon, Inc., nylon, and expanded polytetrafluoroethylene. The suspension suture 12 is preferably a monofilament absorbable suture and the tissue-release suture is preferably a braided suture.

The shafts 14, 15 are preferably at least 4 inches (10 cm) in length, more preferably from about 4 inches to about 15 inches (10 cm to 36 cm) in length, still more preferably from about 5 inches to about 10 inches (13 cm to 25 cm) in length, and most preferably about 6 inches (15 cm) in length. The diameters of the shafts are sufficiently small to allow for percutaneous insertion, and may range from about 0.5 mm to 2.0 mm. The diameters can be approximately the same as those of a typical Keith needle. The insertion or distal end 18, 19 of each shaft is preferably tapered although terminating in a blunt, non-cutting end. The shafts can be of any material of construction that can be sterilized, is non-corrosive, and that might be used in a surgical procedures. Examples are stainless steel and plastics.

The “fusing” of the proximal ends 20, 21 of each shaft to the two suture ends can be achieved by any conventional manufacturing method that results in a permanent joinder, i.e., one that cannot be readily disengaged by manual force short of extreme force, and one that does not involve the tying of knots. The “fusing” may thus be accomplished by a chemical bond such as by the use of an adhesive, or by heat bonding. Alternatively, and preferably, each shaft is swedged on to the sutures in accordance with the typical use of the term “swedged on” in medical procedures. This method of attachment is achieved by using a shaft with a hollow end, inserting both suture ends into the hollow end, and mechanically crimping the hollow end over the inserted sutures to compress the sutures inside the shaft where the sutures will be held by compression and friction force.

The ring-shaped anchor graft 16, which can also be termed a “pledget,” is a ring of non-absorbable material, which can be any of the nonabsorbable materials included in the list of materials presented above, or other materials that are suitable as medical implants. Preferably, the pledget is made of a porous or microporous material such as GORE-TEX, expanded polytetrafluoroethylene, or a polyester mesh. The ring is preferably toroidal in shape but can be flat or rounded (doughnut-shaped). The size and dimensions of the ring are not critical to the invention and can vary. A preferred range for the thickness of the ring is 1-2 mm, and a preferred range for the diameter of the ring is 2.5-4 mm. The central passage or hole 22 through the center of the ring will be large enough to accommodate the suspension suture 12 and the safety suture 17 and preferably have excess room to promote tissue growth through the hole. A preferred diameter range for the hole is 0.5-3 mm. The suspension suture 12 and the safety suture 16 are both threaded through the hole 22.

The device as shown in FIG. 1 is supplied fully assembled and packaged in sterile packaging. The device can be designed and packaged for a single use followed by disposal. The safety suture can be included as an option. Further components that can be supplied with the device as additional options are a modified Keith needle with an eye to be used as a rescue device and a French eye needle for securing the ends of the suspension suture.

To use the device as described above in a surgical procedure for the elevation of the malar fat pads, the incisions are made and the shafts inserted as described above. The orientation of the anchor graft is no longer a critical step of the procedure as it is in the prior art, since the ring-shaped anchor graft 16 is fully functional in all orientations. If the placement of the anchor graft or any of the sutures is deemed undesirable, the graft, sutures, and shafts can be retrieved by pulling them back through the tissues using the safety suture 17 and then repeating the procedure to achieve the desired placement. Once the desired placement is achieved, the shafts 14, 15 are separated from the sutures 12, 13 by cutting the sutures near the proximal ends 20, 21 of the shafts. The tissue-release suture 13 is removed and the suspension suture 12 secured in place at its distal ends to the temporal fascia.

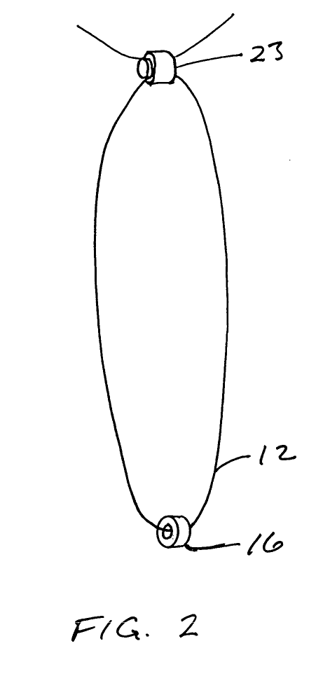

FIG. 2 is an illustration of one method for securing the distal ends of the suspension suture 12. According to this method, the free ends of the suture are passed through a clamp 23, such as a spring-loaded, manually releasable clasp, and the tension adjusted by opening the clamp and either pulling the sutures more tightly by drawing them further through the clamp in the distal direction or relaxing them by allowing them to pull back in the proximal direction.

While the description above applies to a facial suspension to correct midface ptosis, the device described herein can also be used for other soft tissue suspensions in other parts of the anatomy. In general, the foregoing explanation is offered for purposes of illustration. Variations and modifications can be made to the materials used and their shapes and configurations, as well as the procedural steps of their use, without departing from the spirit and scope of the invention.

1. A surgical device for suspending soft tissue to correct midface ptosis, said device comprising:
first and second shafts, each shaft being rigid and straight and each having a distal end and a proximal end,
first and second sutures defined as a suspension suture and a tissue-release suture, respectively, each suture having a first and second end, the first ends of both sutures fused to the proximal end of said first shaft and the second ends of both sutures fused to the proximal end of said second shaft, and
an anchor graft attached to said suspension suture.

2. The surgical device of claim 1 in which said first and second shafts each terminate in a blunt tip.

3. The surgical device of claim 1 in which said proximal end of said first shaft is swedged on to the first ends of both sutures and said proximal end of said second shaft is swedged on to the second ends of both sutures.

4. The surgical device of claim 1 in which said anchor graft is toroidal in shape.

5. The surgical device of claim 1 in which said anchor graft is a microporous fluorocarbon polymer and toroidal in shape.

6. The surgical device of claim 1 in which suspension suture is a monofilament suture, and said tissue-release suture is a braided suture.

7. The surgical device of claim 1 further comprising a manually releasable clamp to receive the first and second ends of one of said sutures.
